(19) United States
(12) Patent Application Publication
(10) Pub. No.: US 2017/0202976 A1
(43) Pub. Date: Jul. 20, 2017

(54) GOLD NANOPARTICLES FUNCTIONALIZED WITH SEMAPHORIN 3F AND PREPARATION THEREOF

(71) Applicants: Mehmet Ali ONUR, Ankara (TR); Gamze TAN, Ankara (TR)

(72) Inventors: Mehmet Ali ONUR, Ankara (TR); Gamze TAN, Ankara (TR)

(21) Appl. No.: 15/328,296

(22) PCT Filed: Jan. 19, 2015
(86) PCT No.: PCT/TR2015/000018

§ 371 Date: Jan. 23, 2017

(51) Int. Cl. A61K 49/00; A61K 9/51

(52) U.S. Cl. CPC A61K 47/48861

(57) ABSTRACT

Present invention is related to gold nanoparticles functionalized with Semaphorin 3F and a preparation method thereof.

### TECHNICAL FIELD

Present invention is related to the gold nanoparticles functionalized (modified) with Semaphorin 3F (Sema 3F) and a preparation method thereof.

### PRIOR ART

Cancer occurs as a result of disruption of the mechanisms regulating normal behavior of cells. Today, cancer is one of the most important health problems in the world. According to data in developed countries, cancer is the second leading cause of death after cardiovascular diseases [1]. According to the research conducted by World Health Organization (WHO) and International Agency for Research on Cancer (IARC), the number of cancer related deaths is projected to increase to 13.1 million and cancer related deaths are predicted to move up to first place by 2030 [2].

Nowadays, in treatment of cancer, usually surgery, radiation therapy and chemotherapy methods are applied either alone or in combination. In the treatment with surgical method, even after removal of a large portion of the tumor tissue, there may remain an amount of tumor cell in media and this situation induces tumor reappearance. Radiotherapy and chemotherapy are implemented as a supportive treatment of surgical operation although they are more efficient in the treatment of tumors at an early stage. In systemic chemotherapy, administering high dose of the antineoplastic drug in order to achieve effective results also harms healthy cells causing side effects such as nausea, vomiting, weakness, hair loss, negatively affects the patient's resistance mechanisms, and can lead to death of the patient by reducing the quality of life. The major limitations in cancer treatment are the non-specific systemic spread of anti-tumor agents, imbalance in the concentration of drug reaching the tumor side and the difficulties faced in monitoring therapeutic response. Also, the fact that a sufficient amount of drug cannot be transferred to the target area leads to serious complications such as the formation of resistance against many drugs.

The limitations of current treatment methods increase the need for alternative methods. Therefore, among the today's cancer treatment approaches, anti-angiogenic effect mechanisms are frequently used. The role and importance of angiogenesis in anti-angiogenic therapy is great.

Pathologic angiogenesis called neovascularization, which provides the oxygen required for growth, invasion and Metastasis of the tumor tissue, nutrients and growth factors, plays a key role in tumor growth. Studies have shown that tumor cell populations without angiogenesis can grow up to no more than 2-3 mm in diameter or 0.5 mm³ volume [3-5]. Tumoral formation, through the chemical signals secreted by endothelial tissue environment, stimulatingly initiates the formation of vessels.

The most important of angiogenic molecules is vascular endothelial growth factor (VEGF) [6]. Today, the basis of anti-angiogenic therapy constitutes VEGF inhibition. The most common method for VEGF inhibition is based on monoclonal antibodies, which are synthetically produced against VEGF. After the approval of the anti-cancer drug Bevacizumab (Avastin®), which is a monoclonal antibody specifically targeting VEGF, in 2004 by U.S. Food and Drug Administration, the studies in this area showed a great increase.

With the treatment methods developed based on the VEGF blockade, generally vessel formation in primary tumor cells is inhibited and tumor tissue is limited without interfering with the present tumor tissue. Also, during the treatment of rapidly spreading cancer types such as lung cancer, the patient experiences a healing process but such treatment sometimes results in sudden death of the patients. The main reason for this may be some deficits existing in the molecular methods of blocking paths of growth factor through antibodies administered in anti-angiogenic therapy

In parallel with recent developments in nanotechnology, through the emerging drug delivery systems based on nanoparticles, cancer therapeutics are targeted to solid tumors and the ability to escape from phagocytotic excretion via reticuloendothelial system (RES) pathways is increased, which allows to enhance the therapeutic effect [7-9]. Under the theoretical conditions, such delivery systems often infiltrate into the blood vessels around the tumor and accumulate in the tumor microenvironment through passive targeting [10, 11]. Besides, by functionalizing the nanotherapeutics so as to include tumor targeting ligands, and binding them via active targeting to tumor cells, nanoparticles can be attached in an active way into solid tumors. Targeted delivery systems of particles release cancer drugs only in the tumoral region, and thus reduce the accumulation of anti-cancer agents in healthy organs. Consequently, these delivery systems improve the reliability of the cancer treatment and increase the relative effect thereof, and thus serve to increase the therapeutic index. From this point of view, nanotechnological approaches demonstrate superiority compared to the conventional therapy in the treatment of cancer.

The development of new blood vessels is a highly critical process in terms of the growth of tumors. For that reason, the process of angiogenesis is desired to be slowed down, or stopped in the treatment of cancer. There are different studies on cancer biology, in which only Sema 3F has been used. It has been mentioned in these studies that Sema 3F inhibits the proliferation of cells in the treatment of cancer.

The recent developments and studies in the field of cancer biology, have shown that especially the class 3 Semaphorins (Sema 3) in the Semaphorin family play a role in the control of VEGF induced angiogenesis and tumor growth [12].

Semaphorin 3 competes with VEGF in order, to bind to the Neuropilins (NRP1 and NRP2), which play an important role in angiogenesis and VEGF signaling. In the studies, it was observed that in lung cancer, Sema 3F gene (3p21.3) has lost and protein localization has shifted from the cell membrane to the cytoplasm streaming. Also, the cytoplasmic localizations of Sema 3F in the cancer cell lines were observed to be significantly associated with high VEGF levels. When VEGF₁₆₅ and Sema 3F are in the same environment, Sema's affinity to Neuropilin (NRP) is more than about 10 times higher than VEGF. Furthermore, when Neuropilin complexes with Plexin, it also activates other molecular pathways and it both inhibits VEGF and can reduce the proliferation more effectively than anti-angiogenic methods.

In a study, it is shown that Sema 3F repels endothelial cells and this tendency of repelling angiogenic branches can inhibit angiogenesis [13]. Indeed, it has been observed that the vascularization around the tumor formation in malignant melanoma cells expressing recombinant Sema 3F is quite low and that metastatic ability of the cells in this tumor mass is highly impaired. The idea that these effects on the metastatic potential of melanoma cells result from the inhibition of angiogenesis and that it affects tumor cells directly has gained weight, Kusy et al. showed that Sema 3F has blocked tumor formation in lung carcinoma cells (NCI-H157) in a rat orthotopic model [14].

In literature, it is observed that the transfection method is generally used for the administration of Semaphorin. After transfection (Transfection is the process of deliberately transferring a gene into nucleus of another cell through plasmid and introducing it into its DNA) of Semaphorin, reduction of cell proliferation is determined. Normally, a healthy person body can secrete the Semaphorin at a certain level but in the angiogenesis process, the secretion of Semaphorin is suppressed, whereby tumor can grow rapidly and spread more easily. Therefore, by transfecting the expression vector of Semaphorin, Semaphorin is desired to be secreted by cells. Thus, various studies have shown reduction in cell proliferation. Semaphorin, which can be secreted from the human body, may be an alternative to anti-cancer drugs used in anti-angiogenic therapy. Because, its side effects will not be as in the anti-cancer drugs.

Semaphorin and VEGF are in competition. In cancer progression, it is observed a decrease in the expression of Semaphorin and an orientation to the cell nucleus in the localization of these proteins. However, it was observed that when Semaphorin 3F bound to the gold nanoparticles (AuNPs) is given exogenously, the balance between Sema 3F and VEGF₁₆₅ is provided again, and as a result of the VEGF₁₆₅ induced vascularization, endothelial cell proliferation is reduced. In this manner, vessel formation was inhibited in the cancer microenvironment.

Today many studies have shown that the transport process performed through nanoparticles for delivery of therapeutic agents to the in vitro and in vivo the target region provides more effective results.

While many materials are widely used for drug delivery systems, gold nanoparticles occupy an important place in this study. Gold has recently become prominent as a drug and gene carrier due to its non-toxic core, its ability to form bio-compatible complex structures by binding various therapeutic agents and biomolecules in a stable way and since its surface features such as charge and hydrophobicity can be adjusted in a monolayer. For that reason, in this study, AuNPs have been used for orienting Sema 3F molecules to the cell structures.

### REFERENCES

1. Greenlee R. T., Murray T., Bolden S., Wingpo C. A. Cancer statistics. Cancer 2000; 50:7-33.
2. International Agency for Research on Cancer (IARC). GLOBOCAN 2008. http://globocan.iarc.fr/3.
3. Folkman J. What is the evidence that, tumors are angiogenesis dependent? J Nat Cancer Inst 1990; 82:4-6.
4. Folkman J. Tumor angiogenesis, in Mendelsohn J., Howley R. M., Israel M. A., et al (eds): The Molecular Basis of Cancer. Philadelphia, Pa., WB Saunders, 1995, pp 206-32.
5. Folkman J. Tumor angiogenesis. Adv Cancer Res 1974; 19:331-58.
6. Özuysal S. Tümöral anjiogenezis. Türk Patoloji Dergisi 2001; 17(3-4):90-3.
7. Folkman J. Angiogenesis and angiogenesis inhibition: An overview. EXS Regul Angiogenesis 1997; 79; 1-7.
8. Papisov, M. I. Theoretical considerations of RES-avoiding liposomes: molecular mechanisms and chemistry of liposome interactions. Ad. Drug Delivery Rev. 1998; 32:119-38.
9. Woodle, M. C. Controlling liposome blood clearance by surface grafted polymers. Ad. Drug Delivery Rev. 1998; 32:139-52.
10. Nafayasu, A., Uchiyama, K., Kiwada H. The size of liposomes: a factor, which affects their targeting efficiency to tumors and therapeutic activity of liposomal antitumor drugs. Ad. Drug 1999; 40:75-87.
11. Maruyama, K., Ishida, O., Takizawa, T., Moribe, K. Possibility of active targeting to tumor tissues with liposomes. Ad. Drug Delivery Rev. 1999; 40:89-102.
12. Neufeld G., Lange T., Varshaysky A., Kessler O. Semaphorin Signaling in Vascular and Tumor Biology Semaphorins: Receptor and Intracellular Signaling Mechanisms. In: Pasterkamp R. J. (ed.): Springer New York; 2007. p. 118-31.
13. Bielenberg D. R., Hida Y., Shimizu A., Kaipainen, A., et al. Semaphorin 3F, a chemorepulsant for endothelial cells, induces a poorly vascularized, encapsulated, nonmetastatic tumor phenotype. J Clin Invest 2004; 114:1260-71.
14. Kusy S., Nasarre P., Chan D., et al. Selective suppression of in vivo tumorigenicity by semaphorin SEMA3F in lung cancer cells. Neoplasia 2005; 7: 457-65.

### BRIEF DESCRIPTION OF THE INVENTION

Present invention discloses a nanoparticle comprising a gold nanoparticle and Semaphorin 3F which functionalizes said nanoparticle. The molar ratio of gold nanoparticle to Semaphorin 3F is from 1:50.

Present invention also discloses a method for the preparation of a nanoparticle comprising a gold nanoparticle and Semaphorin 3F which functionalizes said nanoparticle and said method comprises the following steps:
a. Dissolution of 1-Ethyl-3-(3-dimethylaminopropyl)carbodiimide in 2-(N-morpholino) ethanesulfonic acid solution at pH between 6 to 6.5, and mixing for about 5 minutes,
b. Addition of gold nanoparticle solution to the 1-Ethyl-3-(3-dimethylaminopropyl)carbodiimide solution obtained in step a and mixing for about 30 minutes,
c. Cleaning of the nanoparticles obtained in step b by size exclusion chromatography,
d. Addition of Semaphorin 3F solution and mixing for about 15 minutes,
e. Addition of a blocking agent,
f. After keeping 1 h at room temperature, incubation of the samples overnight at 4° C.,
g. Removing the unbounded parts by centrifugation.

### OBJECT OF THE INVENTION

The object of the invention is to provide gold nanoparticles functionalized with Semaphorin 3F with a great stability, particle size distribution and activity.

Another object of the invention is to direct gold nanoparticles functionalized with Semaphorin 3F (Sema 3F) to cellular targets in human.

Other object of the invention is to use gold nanoparticles (AuNPs) as a platform for Sema 3F send to endothelial cells effectively and create a strong anti-angiogenic effect.

### DESCRIPTION OF THE DRAWINGS

FIG. 1 shows the diagram for steps of functionalization method of the present invention.

FIG. 2 shows schematic view of functionalized gold nanoparticles with protein (Bovine Serum Albumin or Semaphorin 3F) and tetramethylrhodamine-5-carboxamide cadaverine, (TAMRA).

FIG. 3 shows the UV-Vis absorption spectrum of gold nanoparticles (AuNPs) and gold nanoparticles stabilized with polyethyleneglycol (AuNPs@PEG).

FIG. 4 shows TEM views of gold nanoparticles (a, c and d) and size distribution (b).

FIG. 5 shows TEM views of AuNPs@PEG (a, c and d) and size distribution (b).

FIG. 6 shows UV-Vis absorption spectrum of gold nanoparticles labeled with TAMRA and stabilized with polyethyleneglycol (AuNPs@PEG-T), gold nanoparticles labeled with TAMRA, stabilized with polyethyleneglycol and functionalized with BSA (AuNPs@PEG-T&BSA), gold nanoparticles labeled with TAMRA, stabilized with polyethyleneglycol and functionalized with Semaphorin 3F (AuNPs@PEG-T&Sema 3F).

FIG. 7 shows TEM views of AuNPs@PEG-T&BSA (a, c and d) and size distribution (b).

FIG. 8 shows TEM views of AuNPs@PEG-T&Sema 3F (a, c and d) and size distribution (b).

FIG. 9 shows ζ-potential results and size distribution of AuNPs, AuNPs@PEG and Bioconjugates.

FIG. 10 shows gel views of AuNPs modified by PEG and Bioconjugates.

FIG. 11 shows the Fluorescence spectrum of colloidal AuNPs. AuNPs@PEG-T, AuNPs@PEG-T&BSA, AuNPs@PEG-T&Sema 3F.

FIG. 12 shows the view of cellular uptake 24 hours after the interaction of AuNPs@PEG-T conjugates with A549 cells (×20).

FIG. 13 shows the view of cellular uptake 24 hours after the interaction of AuNPs@PEG-T&BSA conjugates with Human Umbilical Vein Endothelial Cells (HUVEC) (×20).

FIG. 14 shows the view of cellular uptake 24 hours after the interaction of AuNPs@PEG-T&Sema 3F conjugates with HUVEC (×20).

FIG. 15 shows, the view of cellular uptake 24 hours after the interaction of AuNPs@PEG-T&Sema 3F conjugates with HUVEC-2 (×20).

FIG. 16 shows the view of cellular uptake 24 hours after the interaction of AuNPs@PEG-T&Sema 3F conjugates with HUVEC-3 (×20).

FIG. 17 shows the comparison of cellular uptake of the functionalized AuNPs. The scale bar is 20 μm.

FIG. 18 shows the effect of VEGF₁₆₅ in (0-16 ng/mL) on endothelial cell proliferation (HUVEC) vs. time graph. Error bars represent the standards error (n=4, *p<0.05, **p<0.01).

FIG. 19 shows the effect of Sema 3F (0-240 ng/mL) on A549 cells. Error bars show the statistical standard error (n=3, **p<0.01).

FIG. 20 shows the effect of Sema 3 F applied following the VEGF₁₆₅ induction (0.1-240 ng/mL) on the endothelial cell proliferation. The HUVEC activated with VEGF₁₆₅ of 10 ng/mL has been taken as a positive control. Error bars show the standard error.

FIG. 21 shows the results of EdU method implemented on HUVEC. Error bars show the standard error.

FIG. 22 shows the results of EdU method implemented on A549 cells. Error bars show the standard error.

FIG. 23 shows the effect of biofunctionalized nanoparticles on the endothelial cell (HUVEC) proliferation (n=3, *p<0.05, **p<0.01). Error bars represent the standard error.

FIG. 24 shows the cytotoxicity values obtained at the end of the 1st day after applying AuNPs@PEG at different concentrations to A549 cells (n=4, **p<0.01). Error bars show the standard error.

FIG. 25 shows the cytotoxicity values obtained at the end of the 1st, 2nd and 3rd day after applying AuNPs@PEG at different concentrations to HUVEC cells (n=4, **p<0.01). Error bars show the standard error.

FIG. 26 shows the graph showing the effect of the bioconjugates on the endothelial cell (HUVEC) viability. Error bars show the standard error. (n=3).

### DETAILED DESCRIPTION OF THE INVENTION

Gold Nanoparticles which are going to be functionalized with Semaphorin 3F can be produced by any method known in the technical field. Turkevich method relates to a simple synthetic method of gold colloids by the treatment of hydrogen tetrachloroaurate (HAuCl₄) with citric acid in boiling water. In the solution of HAuCl₄, addition of reducing agents nucleates the gold particles. Frens method is the most commonly employed aqueous method. It is possible to control the size of AuNPs from 5 to 150 nm by simply varying the reaction conditions.

In one embodiment of the present invention, for the production of AuNPs, Bastus method has been preferred as it allows the production of particles which are fast, have a relatively narrow size distribution and a uniform shape; and also enables the particles to be functionalized by such ways as phase and ligand exchange in the oncoming process thanks to the citrate which is a covering agent bound to the particle surface with an intermediate binding strength. It is considered to use the particles less than 30 nm, and since the Oswald ripening observed in the production process of particularly big particles does not occur in Bastus method, AuNPs have been synthesized allowing the size distribution to be in a narrower range. Gold nano-particles produced with the Bastus method have an average size of about 20 nm.

Synthesized gold nanoparticles can be directly functionalized with Semaphorin 3F or synthesized gold nanoparticles are optionally, coated with a polymer like, polyethyleneglycol (PEG) before functionalization. Coating process which is the stabilization of nanoparticles with a polymer like polyethylene glycol (PEG) prevents some problems e.g. aggregation during functionalization of particles with protein or following steps. Besides, it is advantageous to use biocompatible material for the target cell. The molar ratio of said gold nanoparticle to polymer is preferably 1:25000.

In functionalization process, Gold nanoparticles prepared according to a method known in the art are functionalized with Semaphorin 3F (activation/functionalization process) and preferably, florescent dye; Tetramethylrhodamine-5-carboxamide cadaverine (TAMRA) is added after the addition of Semaphorin 3F. The dye is preferably added to demonstrate binding efficiency of nanoparticles or if desired to be used in the imaging area.

Functionalization (Activation) Process comprises the binding of Semaphorin 3F to the synthesized nanoparticles according to the Bastus method and the strategy is based on the use of a carbodiimide (EDC) to activate the carboxylic group. Activation conditions should be optimized for each particular nanoparticle type. Proteins are built of amino acids, so they have plenty of carboxylic group and amine groups that can be activated and reactive along activation process with EDC.

Functionalization (Activation) Process comprises the following steps basically:
a) Dissolution of 1-Ethyl-3-(3-dimethylaminopropyl)carbodiimide (EDC) in 50 mM 2-(N-morpholino) ethanesulfonic acid solution (MES buffer) at pH between 6 to 6.5; preferably at pH 6.5 and mixing preferably by stirring or shaking for about 5 minutes (Activation Step),
b) Addition of gold nanoparticle solution to the 1-Ethyl-3-(3-dimethylaminopropyl)carbodiimide solution obtained in step a and mixing preferably by stirring or shaking for about 30 minutes,
c) Cleaning of the activated nanoparticles by size exclusion chromatography (SEC) using a prepacked column PD-10 desalting column (GE healthcare) (Filtration step),
d) Addition of Semaphorin 3F solution and mixing preferably by stirring or shaking for about 15 minutes (Functionalization Step),
e) Optionally, addition of fluorescent dye after 15 minutes and mixing preferably by stirring or shaking for about 15 minutes (Labeling Step),
f) Addition of a blocking agent (preferably polyethylene glycol) (Blocking Step),
g) After keeping 1 h at room temperature, incubation of the samples overnight at 4° C.,
h) Removing the unbounded parts of protein, dye (TAMRA) and PEG by centrifugation. With this purpose, AuNPs were cleaned at 14000 rpm for 30 minutes until in the supernatant no dye was detected by fluorescence spectroscopy. Characterization of the bioconjugates by gel electrophoresis, fluorescence spectroscopy, UVNis, TEM, DLS and Z-potential.

Gold particle solution comprises gold nanoparticles produced according to a Bastus method or other method known in the art or gold nanoparticles coated with a polymer like polyethyleneglycol (PEG).

1-Ethyl-3-(3-dimethylaminopropyl)carbodiimide (EDC) rapidly hydrolyzes in aqueous solution so it should be prepared just prior to conjugation. Reaction rate or efficiency is pH dependent. The last pH is going to be 7 or 7.5.

Fluorescent dye is preferably selected from tetramethylrhodamine or tetramethylrhodamine-5-carboxamide cadaverine and stock solution of said dye can preferably be prepared at concentration of 1 mg/mL.

Blocking agent is selected preferably as polyethylene glycol and more preferably as 750 Da methoxy amino polyethylene glycol (MeO-PEG-NH₂). Polyethylene glycol stock solution prepared at concentration 20 mg/mL.

The amounts of the ingredients used in the functionalization process are preferably calculated to be about 7.500.000 EDC, about 50 protein, about 1000 TAMRA, about 25000 PEG per nanoparticle in a molar ratio. Unbounded protein; Semaphorin, dye (TAMRA) and PEG are removed by centrifugation.

To make in vitro analysis to the nanoparticles of the present invention, following nanoparticle formulations are synthesized:
1. Gold nanoparticles (stabilized with citrate according to Bastus method) (AuNPs),
2. Gold nanoparticles stabilized with polyethyleneglycol (AuNPs@PEG),
3. Gold nanoparticles labeled with TAMRA and stabilized with polyethyleneglycol (AuNPs@PEG-T),
4. Gold nanoparticles labeled with TAMRA, stabilized with polyethyleneglycol and functionalized with BSA (AuNPs@PEG-T&BSA),
5. Gold nanoparticles labeled with TAMRA, stabilized with polyethyleneglycol and functionalized with Semaphorin 3F (AuNPs@PEG-T&Sema 3F),
6. Gold nanoparticles stabilized with polyethyleneglycol and functionalized with BSA (AuNPs@PEG&BSA), and
7. Gold nanoparticles stabilized with polyethyleneglycol and functionalized with Semaphorin 3F (AuNPs@PEG&Sema 3F).

### Synthesis of Gold Nanoparticles (AuNPs):

The seeded-growth method of AuNPs synthesis is a procedure, whereby a series of AuNPs samples with varying sizes could be obtained by a single preparation proceeding involving a desired number of growth circles, which are initiated by a common seed generation step.

A solution containing 150 mL (2.2 mM) trisodium citrate dehydrate (Na₃C₆H₅O₇.2H₂O) in a 250 mL flask was heated to 100° C. with stirring under reflux. Using a syringe, 1 mL of 25 mM hydrogen tetrachloroaurate (III) hydrate (HAuCl₄.3H₂O) was injected into the flask and stirred at 100° C. The solution turned deep red. The temperature was reduced to 90° C. and stirred continuously another 30 minutes. Then, 1 mL sodium citrate (60 mM) and 1 mL of HAuCl₄ solution (25 mM) were sequentially injected (time delay 2 minutes). After 30 min, the reaction was cooled down to room temperature. Finally, the resultant solution was filtered through 0.22 μm cellulose nitrate filter. Ultrapure water with a resistance greater than 18.2 MΩcm⁻¹ was used for all experiments. All glassware was cleaned in aqua regia (3:1 HCl/HNO₃) and rinsed with ultrapure water.

Stabilization of Gold Nanoparticles (AuNPs) with Polyethylene Glycol (PEG)

Particles were modified/coated with a heterofunctional α-Mercapto-ω-carboxy PEG chain with a thiol group and a carboxylic group in the other end (3 KDa). For that a gold nanoparticles solution was incubated with 100.000 chains of PEG per nanoparticle, the pH, was rise to 12 with sodium hydroxide (NaOH) (1M) to do the process faster. Afterwards, PEGylated NPs (AuNPs@PEG) were cleaned by centrifugation. Finally characterized Uv-Vis spectra, DLS and TEM.

Biofunctionalization of AuNPs@PEG with Model Protein (BSA) or Sema 3F Labeled with TAMRA

In activation/functionalization process, Semaphorin 3F (Sema 3F) and Bovine serum albumin (BSA) were used. BSA was used as a model protein to generate the positive control groups. Semaphorin 3F is dissolved in phosphate buffer (PBS) pH 7 to 7.5 in the concentration of 50 or 100 μg/mL and BSA stock solution is prepared in the concentration of 2 mg/mL.

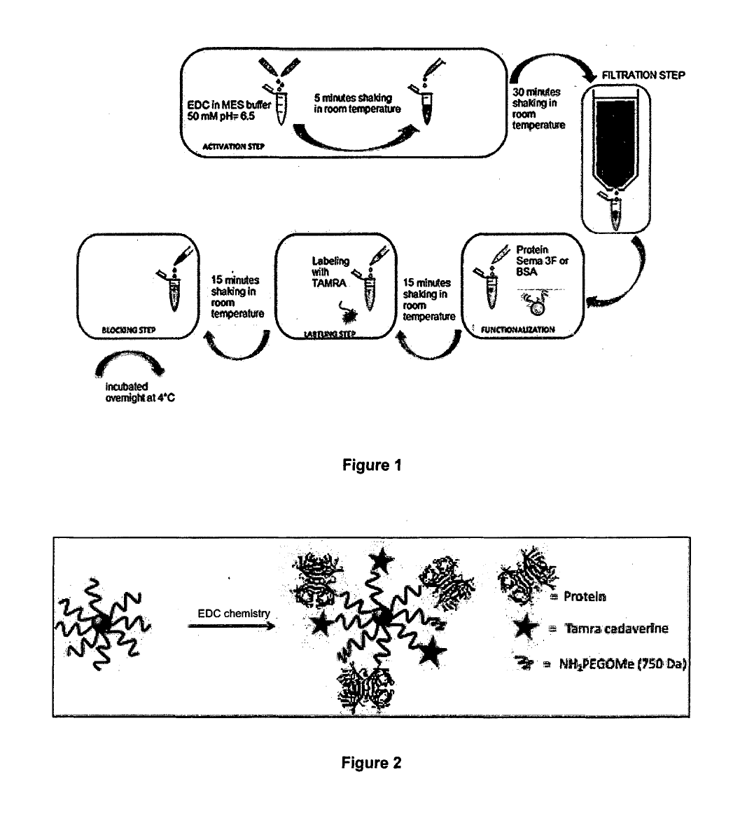

In this method, carbocyclic group on the NPs was activated in a solution of MES (2-(N-morpholino) ethanesulfonic acid) with EDC and at pH 6, and cleaned by size exclusion chromatography (SEC) using a PD-10 salt removing colon. Initially, target proteins BSA or Sema 3F was added followed by addition of TAMRA cadaverine. After a 15 minutes waiting period, blocking agent (NH2-PEG-OMe; 750 Da) was added to mask gold-gold or gold-protein interactions and to prevent precipitation. The samples kept at room temperature for 1 hour were allowed to stay at +4° C. and in a dark environment overnight. At the end of the said time period, excess ligand in biofunctionalized NPs was removed by centrifugation at 14000 rpm for 30 minutes (until no dye molecule was determined in the supernatant by fluorescence spectrometer) and sterilized and made ready for use in cell culture studies (FIG. 1 and FIG. 2). All the reactions during functionalization are performed in a material made of glass.

Production of AuNPs@PEG labeled only with Tamra (AuNPs@PEG-T) was carried out in an identical manner, except that Sema 3F or BSA was not added in the process.

Following experiments are performed on the nanoparticles of the present invention which are produced according to a functionalization method below:

### Characterization of AuNPs and AuNPs@PEG

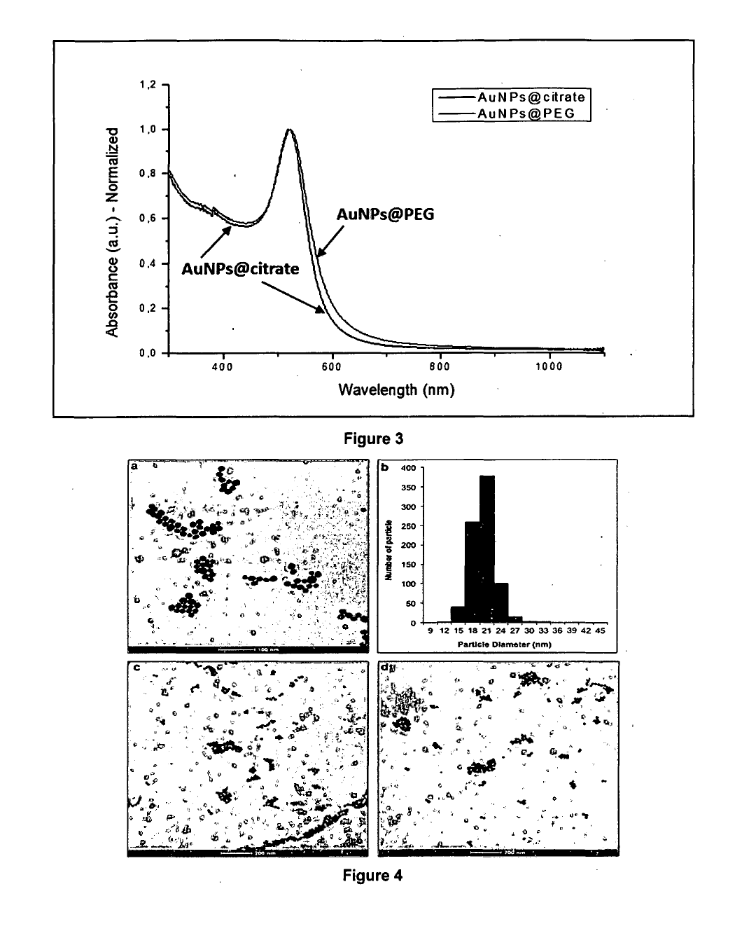

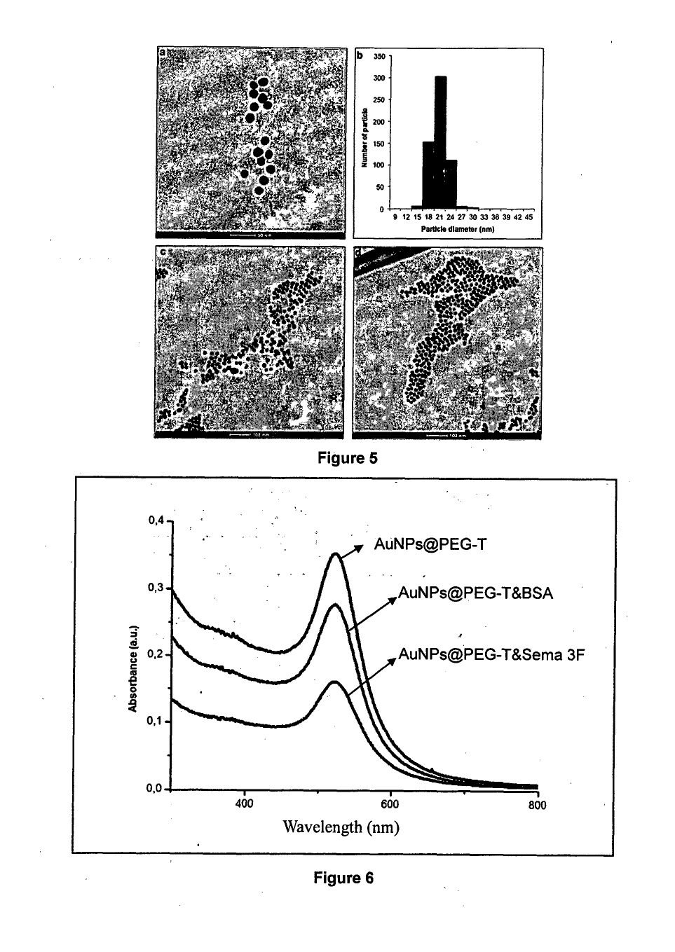

The synthesis of AuNPs according to Bastus Method was performed successfully. The features of synthesized particles; gold nanoparticles (AuNPs) and PEG-stabilized gold nanoparticles (AuNPs@PEG) such as size, shape and surface load were characterized by UV-Vis spectroscopy (FIG. 3), TEM (FIGS. 4a-4d and 5a-5d) and DLS (Table 1). AuNPs of 20 nm reached at 520 nm wavelength surface plasmon resonance, shows maximum absorption value. This results from optic property of nano-sized materials, which is caused by mainly stimulation of electrons of transmission band. When AuNPs (AuNPs@PEG) stabilized with HS-PEG-COOH, i.e. PEGylated, was examined by UV-Vis absorption spectroscopy; it was observed that plasmon absorption peak of AuNPs@PEGs shifted from 520 nm to 522 nm. This shift was caused by the fact that stable Au—S covalent bonds formed between AuNP and HS-PEG-COOH modified dielectric periphery of AuNPs.

AuNPs and AuNPs@PEG with a diameter of about 20 nm were prepared in a narrow size distribution (FIG. 4a-4d and FIG. 5a-5d). Upon an analysis of TEM views, it was determined that the shape of AuNPs is spherical, the average particle size ranges between 12-30 nm, particularly approximately 18.8±2.4 nm (n=798), and the average particle sphericity is 0.90±0.02. With respect to AuNPs@PEG, it was determined that the average particle size ranges between 15-25 nm, particularly approximately 19.3±2.1 nm (n=582) and the average particle sphericity is 0.89±0.02. It is seen that an increase of about 0.5 nm occurred in size. In TEM pictures, it is observed that a clouded layer was available around AuNPs@PEGs, meaning that AuNPs are coated with PEG.

In studies for determining surface charge, it was determined that surface charge of AuNPs is −38.3 mV and that of AuNPs@PEGs is −32.4 mV (Table 1). These results show that gold surface is coated with negative citrate ions and the synthesized NP suspension is stable. The average size of NPs was found to be 25 nm for AuNPs and 39 nm for AuNPs@PEG; whereas polydispersity was 0.188 and 0.240, respectively. Since polydispersity is in the range of 0.08 and 0.7, it is seen that NPs are of almost single distribution.

TABLE 1
DLS results of AuNPs and AuNPs@PEG

| | Z-mean | | | | | ζ-potential |
|---|---|---|---|---|---|---|
| | [nm] | PdI | Number | Intensity | Volume | [mV] |
| AuNPs | 24.99 | 0.183 | 15.22 | 30.52 | 19.87 | −38.3 |
| AuNPs@ | 39 | 0.240 | 23.93 | 50.09 | 32.07 | −32.4 |
| PEG | | | | | | |

Functionalization of AuNPs with Sema 3F and Characterization

AuNPs@PEGs have been functionalized with BSA or Sema 3F through strong covalent bonds by means of the chemical EDC, and labeled by the fluorescent dye TAMRA. The functionalized bioconjugates are characterized by using UV-Vis absorption and fluorescence spectroscopy, DLS, TEM, and gel electrophoresis. AuNPs conjugated with Sema 3F (AuNPs@PEG-T&Sema 3F) were successfully prepared. Furthermore, it has been for the first time that functionalization of a heavy protein like Sema 3F (Semaphorin 3F Fc Chimera˜111.6 KDa) with AuNPs was performed.

During biofunctionalization process performed by using AuNPs@PEG, only those NPs labeled with TAMRA fluorescent dye (AuNPs@PEG-T) were used as a control group. Nanoparticles functionalized with BSA labeled with TAMRA (AuNPs@PEG-T&BSA) and nanoparticles functionalized with Sema 3F labeled with TAMRA (AuNPs@PEG-T&Sema 3F) are prepared.

UV-Vis spectrum of NPs (AuNPs@PEG-T&BSA, AuNPs@PEG-T&Sema 3F) functionalized with BSA and Sema 3F was measured. It was found that λₘₐₓ value for AuNPs@PEG-T 522 nm and for all bioconjugates (AuNPs@PEG-T&BSA, AuNPs@PEG-T&Sema 3F) were 523 nm. The resulting graph was as given in FIG. 6. UV-Vis results of AuNPs functionalized with BSA and Sema 3F are shown in the following table 2. Since AuNPs are stabilized with PEG, all bioconjugates in which ligand parts directly interacts with NP surface display a plasmon wavelength similar to AuNPs@PEG.

TABLE 2

| | Absorbance |
|---|---|
| Sample | value λₘₐₓ |
| AuNPs@PEG-T | 522 nm |
| AuNPs@PEG-T&BSA | 523 nm |
| AuNPs@PEG-T&Sema 3F | 523 nm |

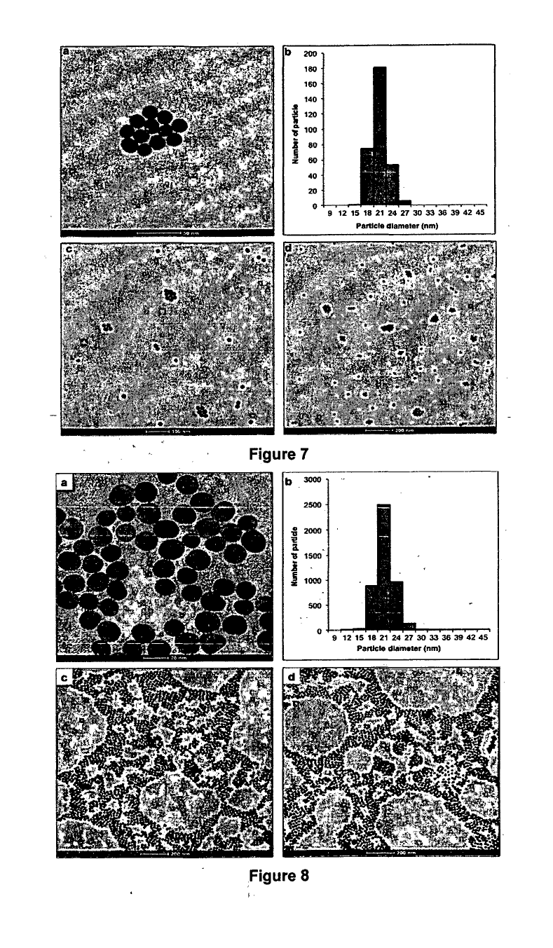

AuNPs@PEG-T&BSA and AuNPs@PEG-T&Sema 3F bioconjugates are stable in the buffer solution that is used, and no big agglomeration has been formed by them. This is clearly seen in the histogram of size distribution and on TEM views (FIG. 7a-7d).

In FIG. 7b, upon analyzing three TEM views for AuNPs@PEG-T&BSA, it was determined that average particle size is about 19.4±1.9 nm (n=316), and the average particle sphericity is 0.91±0.03.

In FIG. 8a-8d, upon analyzing three TEM views for AuNPs@PEG-T&Sema 3F, it was determined that average particle size is about 19.90±2.8 nm (n=4604), and the average particle sphericity is 0.90±0.03.

The changes of hydrodynamic radius as well as the other physicochemical properties of AuNPs, AuNPs@PEG and Bioconjugates are shown in table 3 below.

TABLE 3
Summary of the physicochemical properties of AuNPs

| | | Hydrodynamic | | |
|---|---|---|---|---|
| | Metallic | radius | Net | |
| | core | (Z-mean) | Load | Number |
| | [nm] | [nm] | [mV] | [nm] |
| AuNPs | 18.8 ± 2.1 | 24.99 | −38.3 | 15.23 |
| AuNPs@PEG | 19.3 ± 2.4 | 39 | −32.4 | 23.93 |
| AuNPs@PEG-T | | 39.42 | −23.2 | 28.22 |
| AuNPs@PEG-T&BSA | 19.4 ± 1.9 | 42.81 | −11.4 | 29.03 |
| AuNPs@PEG-T&Sema | 19.9 ± 2.8 | 64.40 | −9.81 | 29.57 |
| 3F | | | | |

According to the results, the following hydrodynamic diameters were found: 39 nm for AuNPs@PEG, 39.42 nm for AuNPs@PEG-T, 42.81 nm for AuNPs@PEG-T&BSA and 64.40 nm for AuNPs@PEG-T&Sema 3F. In the results for AuNPs@PEG, when the surface charge that was −32.4 mV was compared with AuNPs@PEG-T, a difference of −9 mV was observed. The surface charge of AuNPs@PEG-T&BSA was found to be −11.4 mV, whereas the surface charge of AuNPs@PEG T&Sema 3F was −9.81 mV. It is, seen that the surface charge of NPs functionalized with BSA and Sema 3F has increased. It is believed that the increase in the surface charge is caused by modifications on NP surface experienced after covalent binding of BSA and Sema 3F with EDC chemical.

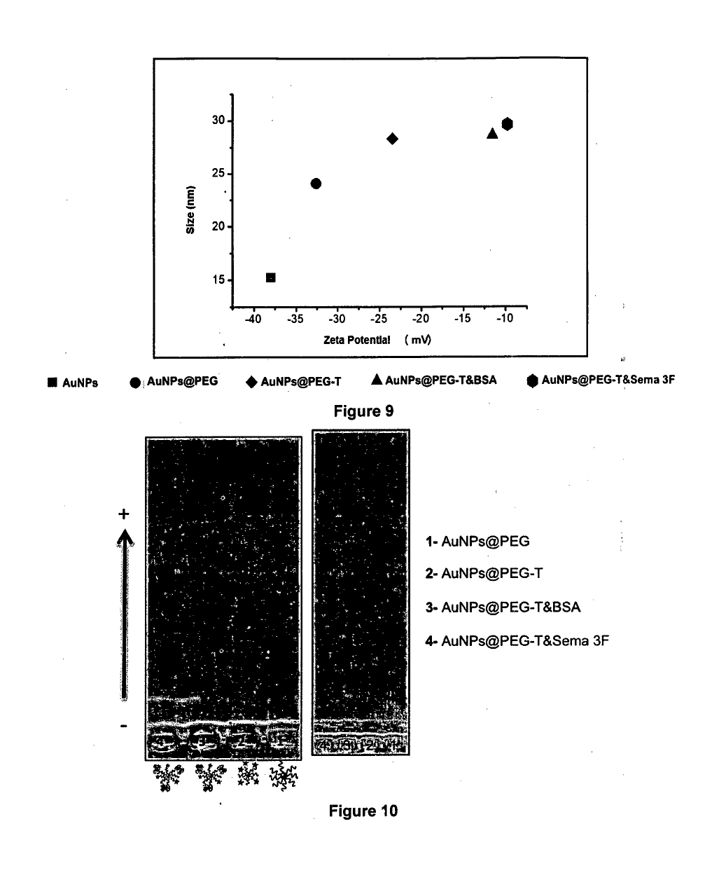

Furthermore, the increases in size and ζ-potential distribution of AuNPs, AuNPs@PEG and Bioconjugates are shown, in FIG. 9.

FIG. 10 shows gel views of AuNPs modified by PEG and Bioconjugates. e.g. agarose-gel electrophoresis. Gel electrophoresis was used to qualitatively examine net charge and EDC combination products among the resulting colloids. The pictures of gel electrophoresis of four different samples, after biofunctionalization, performed in 1% agarose gel for 30 min. (left) and 60 min. (right) are shown. It is observed that AuNPs@PEG is the most rapid one whereas other bioconjugates, when compared with AuNPs@PEG, are very slow and they even do not progress, and no significant difference is available between them. When compared with AuNPs@PEG, the delay of biofunctionalized NP is an indicative that biofunctionalization process is substantially successful.

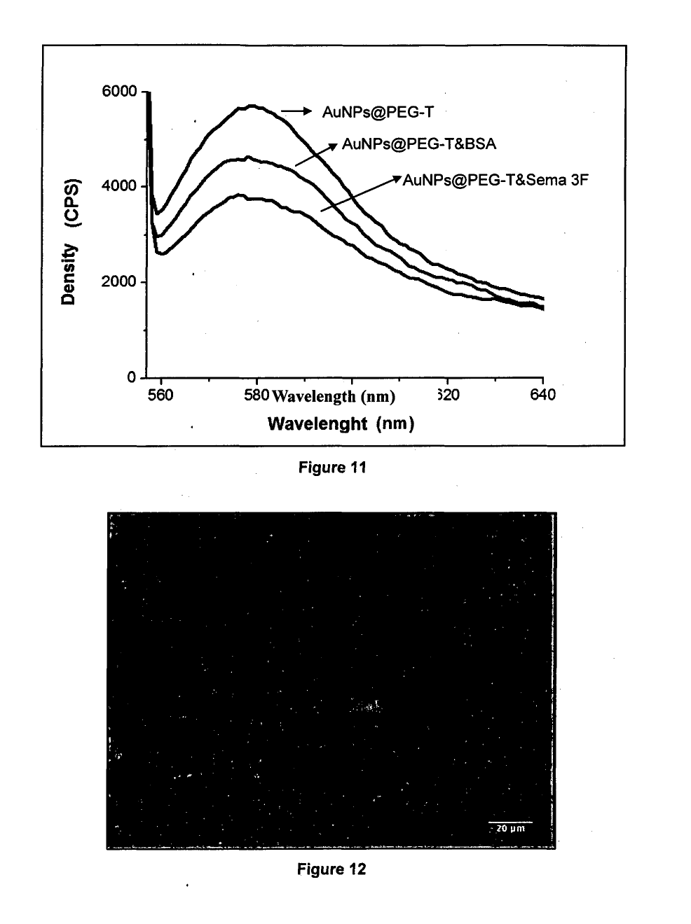

FIG. 11 shows the fluorescence spectrum of colloidal AuNPs. AuNPs@PEG-T, AuNPs@PEG-T&BSA and AuNPs@PEG-T&Sema 3F. It is seen that the florescence intensity of AuNPs@PEG-T is greater than that of those functionalized with BSA and Sema 3F. Here, it is observed that the florescence intensity decreases due to suppression property of florescence. These results show that AuNPs dye hybrids resulting froth functionalization of biomolecules may be used as very sensitive viewing probes and allow examination thereof by microscopy or spectroscopy techniques.

### Stability of AuNPs

In order to determine aggregation of AuNPs@PEGs, DLS is employed. For purposes of showing the spherical stability of AuNPs, size distribution of AuNPs@PEGs in medium with serum and serum-free medium at 37° C. is shown and characterized. The measurements were carried out in triplicate.

The measurement results of DLS were 39.45±0.38 nm in AuNPs@PEG aqueous medium, 37.54±1.3 nm in medium with serum, and 40.49±1.37 nm in serum-free medium. It was shown that AuNPs were more stable in physiological conditions, after being modified with PEG. In order to evaluate cytotoxicity of NPs in culture medium and physiological conditions and to understand interaction of NPs with biological systems, significance of NP characterization was shown. Table 4 shows the stability of AuNPs@PEG.

TABLE 4
Results of AuNPs@PEG stability

| | | | Serum-free |
|---|---|---|---|
| | AuNPs@PEG | Medium with serum | medium |
| hydrodynamic | 39.45 ± 0.38 | 37.54 ± 1.3 | 40.49 ± 1.37 |
| diameter (dh), | | | |
| nanometer (nm) | | | |

### Evaluation of Cellular Uptake

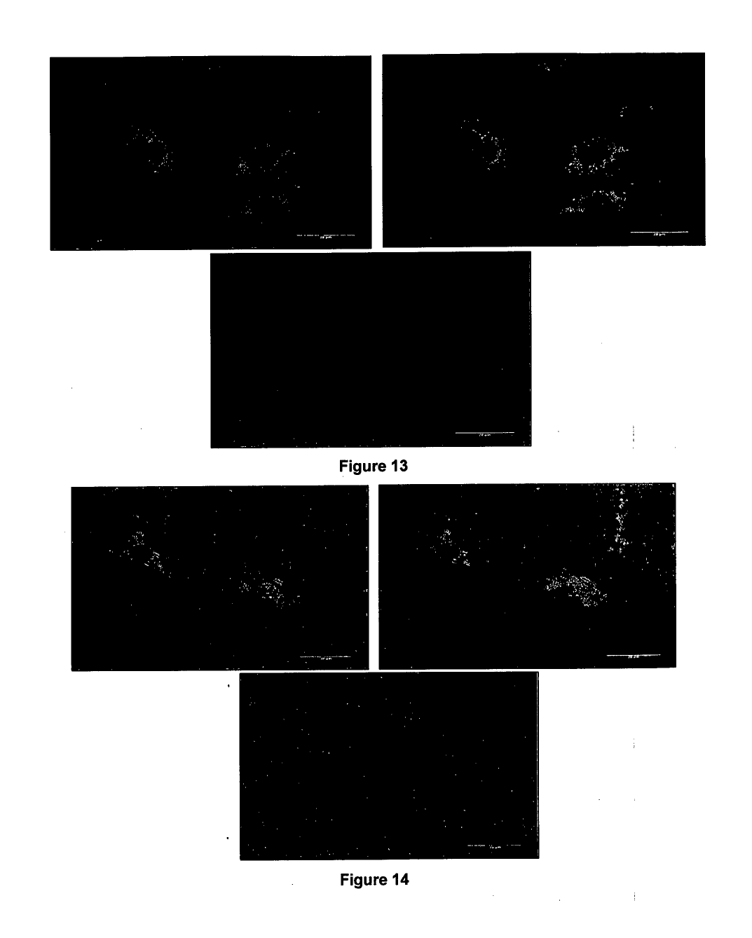

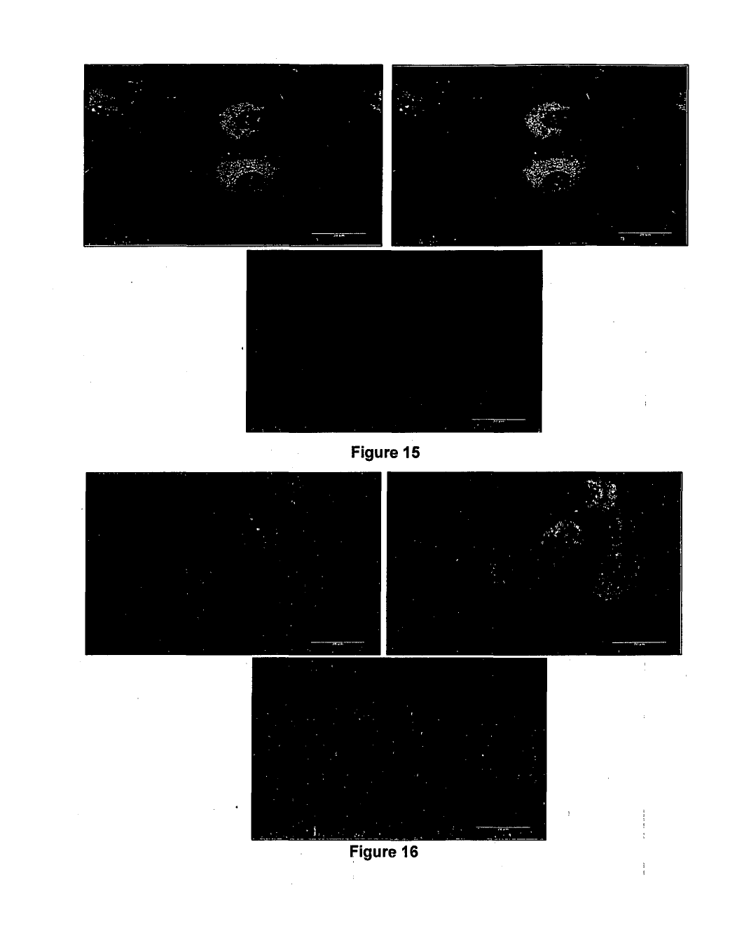

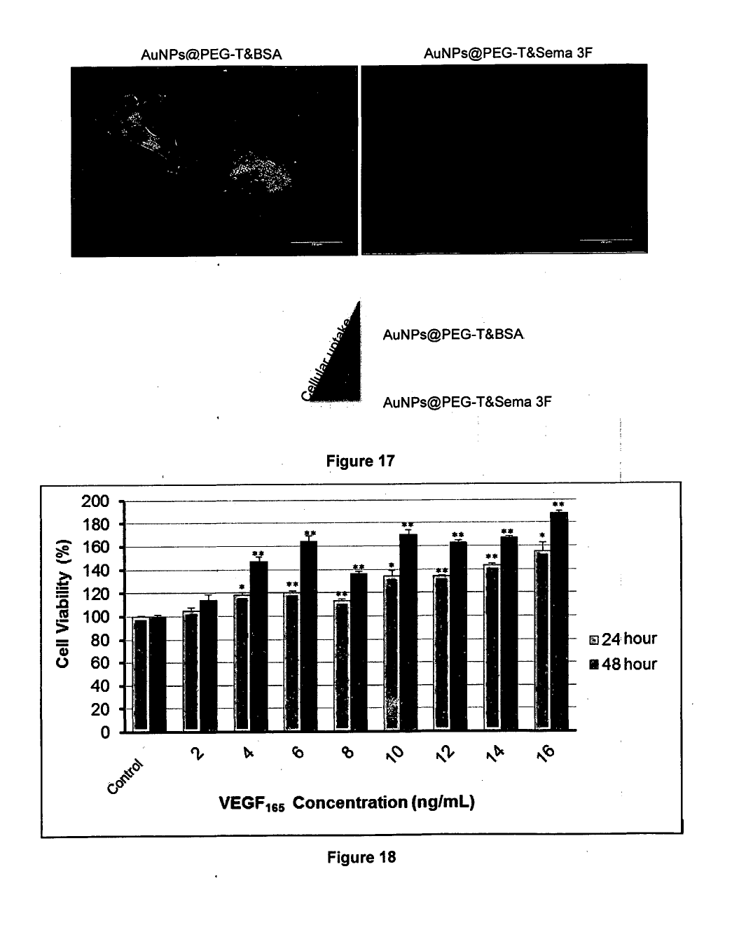

The cells incorporate colloidal NPs via specific or non-specific interaction through receptor-ligand interaction. The object is to transfer the molecules adsorbed on gold nanoparticle surface into Cells. The ability of AuNPs modified with PEG (AuNPs@PEG) and functionalized with proteins (AuNPs@PEG-T&BSA, AuNPs@PEG-T&Sema 3F) to penetrate into cells is examined. It is seen that bioconjugates are received in the cells through receptor mediated endocytosis. The fact that bioconjugates are labeled with TAMRA also enables displaying their intake in the cells. When AuNPs@PEG-T&Sema 3F conjugates are compared with AuNPs©PEG-T&BSA, it is seen that they have been internalized more actively by the cells (FIG. 17a-17c). Of course, cellular uptake mechanism varies for different gold nanoconjugation classes due to different; surface chemistry, size and charge. Displacement reactions may be used in regulating the ability of AuNPs to be internalized by the cells (FIG. 12-17).

### Cell Proliferation

### Forming a Suitable Angiogenesis Model

Among the pro-angiogenic factors, VEGF is the most prominent factor. In this study, a VEGF₁₆₅ concentration required to form a suitable in vitro angiogenesis model by inducing the endothelial cell proliferation was determined as 10-16 ng/mL.

In order to make a more precise determination for VEGF₁₆₅ amount required for inducing angiogenesis formation, endothelial cells were incubated in VEGF₁₆₅ of different concentrations from 0 to 16 ng/mL for 24 hours and 48 hours. Accordingly, it was found that cell proliferation is significantly high in VEGF concentrations of 4 ng/mL and more, when compared to the control group (p<0.05). It has been found suitable that VEGF₁₆₅ amount required for vascularization formation on endothelial cells (HUVEC) is in the range of 10-16 ng/mL. FIG. 18 shows the effect of VEGF₁₆₅ in (0-16 ng/mL) on endothelial cell proliferation (HUVEC) vs. time graph.

### Evaluation of the Effect of Sema 3F on Cell Proliferation

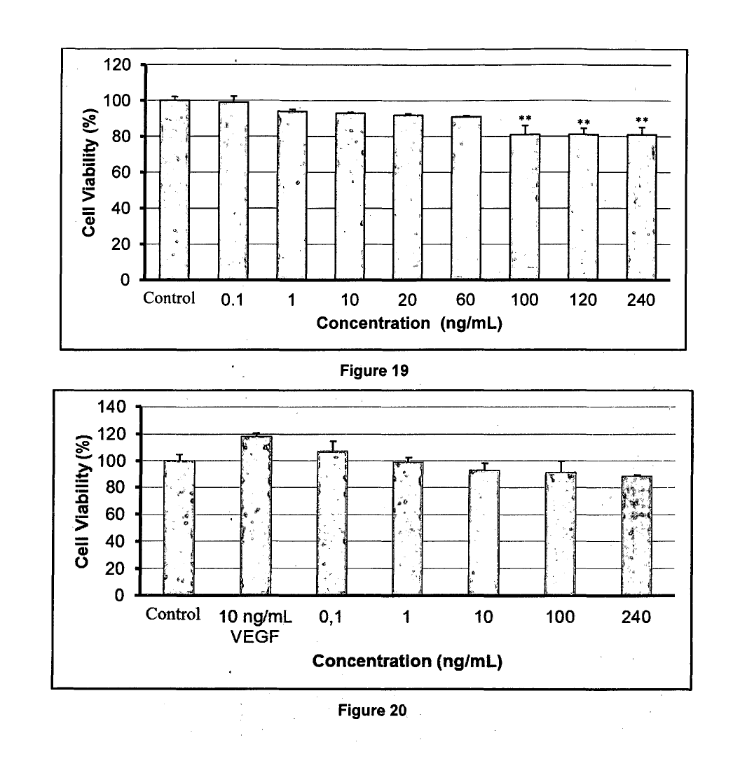

In order to evaluate the tumor suppressor effect of Sema 3F, the effect of its different concentrations in the range of 0-240 ng/mL (0.1, 1, 10, 20, 60, 100, 120 and 240 ng/mL) on A549 cells was examined (FIG. 19). According to the cell proliferation graph, Sema 3F showed its effect at the concentrations of 1 ng/mL and above 1 ng/mL. Furthermore, Sema 3F showed A549 cell proliferation inhibiting effect at the concentration levels of 100 ng/mL and above 100 ng/mL. The efficient value to stop tumoral cell proliferation was observed to be 100 ng/mL.

It has been found out that different concentrations of Sema 3F caused a meaningful difference on the cell proliferation (p<0.05). Similar to the cell proliferation graph, when the concentrations equal to and above 100 ng/mL are compared with the control group, a meaningful decrease has been observed in the A549 cell proliferation (p<0.01).

FIG. 20 shows the effect of Sema 3F (0.1-240 ng/mL) applied following the 10 ng/mL VEGF₁₆₅ induction on the endothelial cell proliferation. The HUVEC activated with VEGF₁₆₅ of 10 ng/mL has been taken as a positive control. Error bars show the standard error. 100 ng/mL or 240 ng/mL decreased cell proliferation significantly.

### Evaluation of the Effect of Sema 3F by Click-EdU on Cell Proliferation

The effect of Sema 3F on proliferation of cells has been performed by the methods of Click-EdU. As a result, it has been shown that Sema 3F has reduced proliferation of both VEGF-induced endothelial cells and A549 cells.

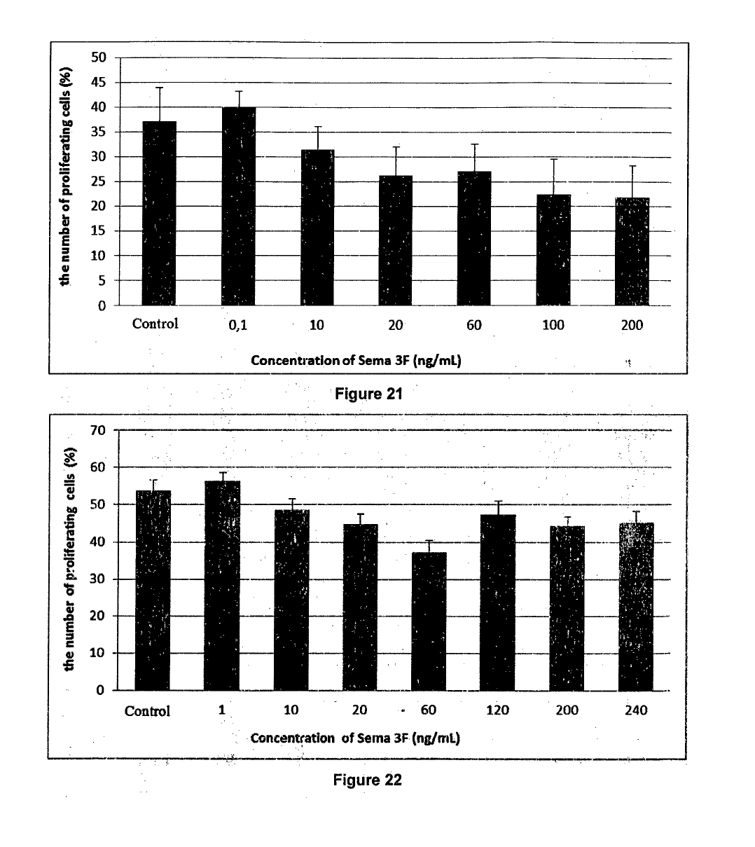

FIG. 21 shows the results of EdU method implemented on HUVEC. The effects of directly administered Sema 3F on HUVEC proliferation for the concentrations of 0, 1, 10, 20, 60, 100 and 200 ng/mL were evaluated All groups other than the control group were incubated with 12 ng/mL VEGF₁₆₅ prior to EdU. Especially 100 ng/mL and higher concentrations Sema 3F showed significantly decrease of cell proliferation. These results suggest that MTT results.

FIG. 22 shows the results of EdU method implemented on A549 cells. The effects of Sema 3F on A549 cells for the different concentrations of 1, 10, 20, 60, 120, 200 and 240 ng/mL were evaluated. 60 ng/mL Sema 3F was found to be most effective for reducing cell proliferation.

Evaluation of the Effect of the AuNPs Conjugated with Sema 3F

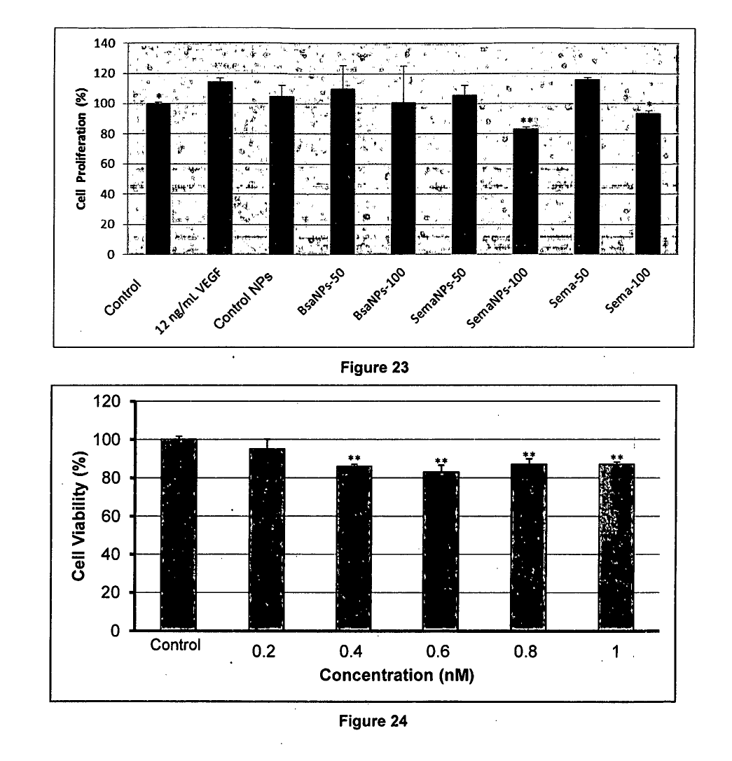

In order to evaluate the effect on angiogenesis, the effect of different groups on VEGF₁₆₅ (12 ng/mL) induced endothelial cell (HUVEC) proliferation was examined (FIG. 23).

Following groups are examined:
only endothelial cell (HUVEC) as control group,
the group applied with only 12 ng/mL VEGF₁₆₅, and with no nanoparticle as 12 ng/mL VEGF group,
the group applied AuNPs@PEG labeled with TAMRA (AuNPs@PEG-T) as control NPs,
the group applied AuNPs@PEG functionalized with 50 ng/mL BSA and labeled with TAMRA (AuNPs@PEG-T&BSA) named as BsaNPs-50,
the group applied AuNPs@PEG functionalized with 100 ng/mL BSA and labeled with TAMRA (AuNPs@PEG-T&BSA) as BsaNPs-100,
the group applied AuNPs@PEG functionalized with 50 ng/mL Semaphorin 3F and labeled with TAMRA (AuNPs@PEG-T&Sema 3F) named as SemaNPs-50,
the group applied AuNPs@PEG functionalized with 100 ng/mL Semaphorin 3F and labeled with TAMRA (AuNPs@PEG-T&Sema 3F) named as SemaNPs-100,
the group applied 50 ng/mL Semaphorin 3F named as Sema-50
the group applied 100 ng/mL Semaphorin 3F named as Sema-100

All groups, except for the control group, were incubated for 24 hours with VEGF₁₆₅ of 12 ng/mL before the application. Comparison was made based on VEGF₁₆₅ of 12 ng/mL.

Here the effects of nanoparticles of the invention, i.e. AuNPs@PEG-T&Sema 3F were higher than those using only Sema 3F and others. This results show that gold nanoparticles may be used as carriers while increasing the effect of Sema 3F. A small amount of Sema 3F may be coupled to gold nanoparticles and dispatched so as to obtain an anti-proliferative effect, and cell proliferation that is highly significant in angiogenesis may be stopped. In this manner, formation of angiogenesis which is the early stage of cancer may be prevented.

In this study, with a carriage performed by gold nanoparticles for tumor angiogenesis inhibition, accumulation of Sema 3F in the target region was accomplished and it was demonstrated that therapeutic effect of Sema 3F is increased with an increase in the concentration of the region. Thus, endothelial cell proliferation was significantly prevented by AuNPs@PEG-T&Sema 3Fs. A large biologic molecule such as Sema 3F which is for the first time be secreted from normal cells and actuating some molecular paths, and having anti-angiogenic effects was conjugated with AuNPs@PEG instead of a targeting peptide or an anti-cancer medicament that may provide a dose-dependent toxic effect, to thereby minimize possible side effects. In this way, future studies are likely to enable that it may be used as a medicament for cancer treatment.

### Cytotoxicity of AuNPs@PEG

Different concentrations of sterilized AuNPs@PEGs in cell culture medium were prepared. For such purposes, 96-well culture plate was used. The cells in the amount of 5000 cell/mL were seeded in each well. The culture medium on the cells was removed and AuNPs@PEG were incubated in medium with A549 cells for 24 hours; and on Human Umbilical Vein Endothelial Cells (HUVEC) for 24 hours, 48 hours and 72 hours, with a medium comprising AuNPs@PEG of different concentrations (0.2 nM, 0.4 nM, 0.6 nM, 0.8 nM and 1 nM) and at 37° C. under 5% CO₂ condition.

In the medium, NP-free group was used as a control group. After the incubation period, the culture medium in all wells was removed, and dissolved in 100 μL medium for MTT test, and a solution of 10 μL MTT (3-[4,5-dimethylthiazol-2-yl]-2,5-diphenyltetrazolium bromide; thiazolyl blue) was added to each well. The 96-well culture plate was incubated at 37° C. under 5% CO₂ condition for 4 hours, and 100 μL dissolution solution was added to each well and sample measurements were performed at 570 nm.

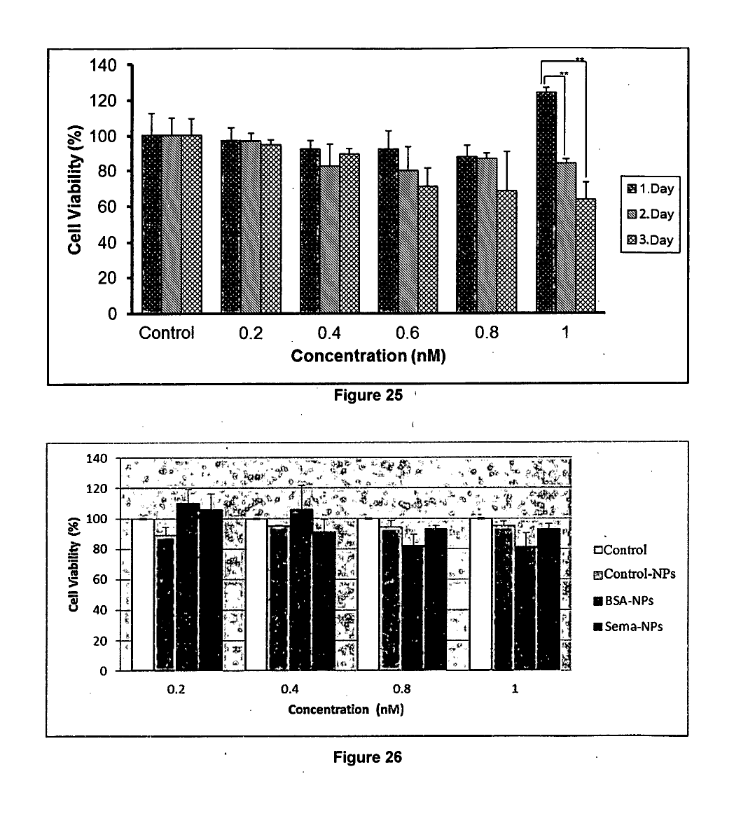

Cell viability values (%) obtained upon evaluation of the cytotoxicity of AuNPs@PEG formulations in vitro at a concentration range of 0-1 nM are given in FIG. 24 (On the cell A549 cells) and FIG. 25 (HUVEC endothelial cells). The experiments were repeated four times both.

It has been found that nanoparticle formulation of the present invention does not cause any toxic effect. The results in FIGS. 24 and 25 show that NPs do not damage mitochondrial respiration (as cell viability does not decrease below 80%).

### Evaluation of Cytotoxicity of Functionalized AuNPs

In order to evaluate, cytotoxic effect of bioconjugates, HUVECs—with the culture medium thereon was removed—were incubated with bioconjugates of different concentrations. The effects of the four different concentrations, i.e. 0.2 nM, 0.4 nM, 0.8 nM and 1 nM, of the bioconjugates AuNPs@PEG-T (control NPs), AuNPs@PEG-T&BSA and AuNPs@PEG-T&Sema3F on cell viability were compared with the control group (FIG. 26).

In the results shown in FIG. 26, it is seen that all bioconjugates are not cytotoxic, in concentrations employed in the experiment, and the cell viability is above 80%. Taking into account of, general studies, cytotoxicity of AuNPs may vary depending on the size, shape of NPs, surface modification, chemical components of surface ligands and cell type. Although cancer cells are used in most of the cytotoxicity studies performed in vitro medium, endothelial cells (HUVEC) which are not commonly studied and which are highly sensitive are used in this study. Bioconjugates had a small, effect on the cell viability on these sensitive cells, but Increased the effect of lowering cell proliferation. This indicates that in the future bioconjugates may be successfully used in treatment of a number of diseases using nanocarrier systems.

Accordingly, in addition to inhibiting VEGF typically provided by anti-VEGF monoclonal antibody the proliferation of tumor cells, which play a role in the differentiation and survival remain with antimetastatic and anti-angiogenic effects of Semaphorin 3F targeting endothelial cells by binding covalently to gold nanoparticles and as a result of the reduction in effective cell proliferation was aimed at preventing the spread of cancer.

Through a combined approach including of Inhibition of VEGF₁₆₅ and Sema 3F up-regulation found an effective way to treat cancer. By modifying the synthesized AuNPs with PEG (AuNPs@PEG), they have been ensured to be more stable and the surface required for simplifying the surface functionalization has been prepared.

Gold nanoparticles didn't functionalize with ionic methods. In ionic methods generally two different molecules mix through electrostatic interaction is the adsorption of biomolecules onto the particles in question. Nanoparticle-protein bio-complex prepared by this binding method (ionic) is very sensitive to the environmental condition (pH). The disadvantage is different molecules accumulate on nanoparticles. So it effect colloidal stability negatively and occur aggregation. In the present invention study, we used covalent binding method, which used strong bonds.

Chemical interactions affect the bioactivity of the protein. If the structure of proteins destroyed it cannot show the effect. As a result of chemical binding protein demonstrated effects without disrupting the structure of biomolecules.

With the present invention, it is shown that Sema 3F functionalized with AuNPs@PEG could stop the proliferation of cells more efficiently when the effects of only Sema3F and the Sema 3F functionalized with AuNPs@PEG on proliferation of cells were compared.

When administered directly into the blood path, Sema 3F shows a non-specific distribution. However, it aggregates on a target area by the nanoparticular systems. In this manner, both the dosage may be decreased and its effect can be enhanced. Tumoral growth and angiogenesis is suppressed more efficiently by using bioconjugates of the present invention compared to the single use of Sema 3F.

As a carrier nanosystem, gold nanoparticles functionalized with Semaphorin 3F provides specific accumulation of the required target area (tumor) with active targeting. Thus, as a result of using less amount of Semaphorin, more efficient result, extension quality of life and lifetime of patient can be provided.

The important point in this study is that Sema 3F has been functionalized with AuNPs@PEG as a carrier nanosystem for the first time, and much more decrease has been observed in proliferation of VEGF-induced endothelial cells by active targeting when used together with NPs. This situation shows that its effect can further be increased when Sema 3F is used with NPs. On the other hand, when Sema 3F is administered directly into the blood path without using nanoparticular systems, it distributes on the target non-specifically. Therefore, the AuNPs@PEGs functionalized with Sema 3F in order to slow down the process of angiogenesis when tumors begin to grow, can be used as a medicament in the future as they show enhanced effect compared to the normal. Furthermore, with this method, possible side effects of drugs are reduced through only targeting because both the dosage of the drug can be decreased and its effect can be enhanced in this manner.

In the study, the use of AuNPs draws attention to the nanotechnological treatment methods that may be used except the other methods yielding more effective results. Along with such known anti-angiogenic treatments, cancer can be treated in a more effective and efficient way, and thus the quality of life and the lives of patients can be improved.

In this way, when compared with the known anti-angiogenic cancer therapy, present invention provides more efficient and effective treatment of cancer and the patient's quality of life and life expectancy will be increased. The use of gold nanoparticles with the present invention also draws attention to nanotechnology treatment methods that can be presented as an alternative to transfection methods.

Other active ingredients known in the art can also be functionalized or loaded to the nanoparticles of the present invention like anti-cancer agents.

The nanoparticles of the present invention can be administered in a pharmaceutical composition preferably with pharmaceutically acceptable carriers. Administration of the active compounds can be effected by any method which enables delivery of the compounds to the site of action (e.g., cancer cells) or systemically. These methods include parenteral injection (including intravenous, subcutaneous, intramuscular, intravascular or infusion), oral routes, intraduodenal routes, topical administration, and other delivery routes known from the prior art.

The gold nanoparticles may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository or other dosage forms known from the prior art. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

1. A nanoparticle comprising a gold nanoparticle and Semaphorin 3F which functionalizes said nanoparticle.

2. The nanoparticle according to claim 1 characterized in that the molar ratio of gold nanoparticle to Semaphorin 3F is from 1:50.

3. The nanoparticle according to claim 1 characterized in that said nanoparticle comprises a polymer layer between Semaphorin 3F and said gold nanoparticle.

4. The nanoparticle according to claim 3 characterized in that said polymer is selected from polyethylene glycol.

5. The nanoparticle according to claim 3 characterized in that the molar ratio of said gold nanoparticle to polymer is 1:25000.

6. The nanoparticle according to claim 1 characterized in that said nanoparticle is labelled with a fluorescent dye.

7. The nanoparticle according to claim 6 characterized in that said fluorescent dye is selected from tetramethylrhodamine or tetramethylrhodamine-5-carboxamide cadaverine.

8. The nanoparticle according to claim 6 characterized in that the molar ratio of said gold nanoparticle to said fluorescent dye is 1:1000.

9. A method for the preparation of a nanoparticle according to claim 1 comprises the following steps:
a. Dissolution of 1-Ethyl-3-(3-dimethylaminopropyl)carbodiimide in 2-(N-morpholino) ethanesulfonic acid solution at pH between 6 to 6.5 and mixing for about 5 minutes,
b. Addition of gold nanoparticle solution to the 1-Ethyl-3-(3-dimethylaminopropyl)carbodiimide solution obtained in step a and mixing for about 30 minutes,
c. Cleaning of the nanoparticles obtained in step b by size exclusion chromatography,
d. Addition of Semaphorin 3F solution and mixing for about 15 minutes,
e. Addition of a blocking agent,
f. After keeping 1 h at room temperature, incubation of the samples overnight at 4° C., and
g. Removing the unbounded parts by centrifugation.

10. The method for the preparation of the nanoparticle according to claim 9 wherein 1-Ethyl-3-(3-dimethylaminopropyl)carbodiimide dissolved in step a has the molar ratio of 1:7.500.000 to nanoparticle.

11. The method for the preparation of the nanoparticle according to claim 9 wherein Semaphorin 3F added in step d has the molar ratio of 1:50 to nanoparticle.

12. The method for the preparation of the nanoparticle according to claim 9 characterized in that 2-(N-morpholino) ethanesulfonic acid solution is in the concentration of 50 mM.

13. The method for the preparation of the nanoparticle according to claim 9 characterized in that gold nanoparticle in step 3 is coated with a polymer.

14. The method for the preparation of the nanoparticle according to claim 13 characterized in that said polymer is polyethylene glycol.

15. The method for the preparation of the nanoparticle according to claim 13 characterized in that the molar ratio of the gold nanoparticle to said polymer is 1:25000.

16. The method for the preparation of the nanoparticle according to claim 9 characterized in that a fluorescent dye is added to the solution obtained in step d and mixed for about 15 minutes.

17. The method for the preparation of gold nanoparticles according to claim 16 characterized in that said fluorescent dye is selected from the group of tetramethylrhodamine or tetramethylrhodamine-5-carboxamide cadaverine.

18. The method for the preparation of the nanoparticle according to claim 16 characterized in that the molar ratio of the gold nanoparticle to said fluorescent dye is 1:1000.

19. The method for the preparation of gold nanoparticles according to claim 16 characterized in that said blocking agent is polyethylene glycol.

20. The method for the preparation of gold nanoparticles according to claim 9 characterized in that Semaphorin 3F solution comprises Semaphorin 3F in the concentration of 0-500 μg/ml.

21. The method for the preparation of gold nanoparticles according to claim 20 characterized in that Semaphorin 3F is in the concentration of 100 μg/ml.
